(19) United States
(12) Patent Application Publication
(10) Pub. No.: US 2021/0309594 A1
(43) Pub. Date: Oct. 7, 2021

(54) COMPOSITION COMPRISING 1-CHLORO-2,2-DIFLUOROETHANE AND 1,1-DICHLOROETHYLENE

(71) Applicant: Arkema France, Colombes (FR)

(72) Inventors: Dominique GARRAIT, Charly (FR); Camille SCHERPEREEL, Bourgoin Jallieu (FR)

(73) Assignee: Arkema France, Colombes (FR)

(21) Appl. No.: 16/334,458

(22) PCT Filed: Oct. 9, 2017
(86) PCT No.: PCT/FR2017/052751

§ 371 Date: Mar. 19, 2019

(30) Foreign Application Priority Data
Oct. 12, 2016 (FR) 1659845

(51) Int. Cl. C07C 17/20; C07C 17/25; C07C 17/383

(52) U.S. Cl. CPC C07C 17/206; C07C 19/12; C07C 17/383; C07C 17/25

(57) ABSTRACT

Azeotropic and quasi-azeotropic compositions comprising 1-chloro-2,2-difluoroethane and 1,1-dichloroethylene. Also, a method for producing compositions comprising 1-chloro-2,2-difluoroethane and 1,1-dichloroethylene. The composition may include from 1 mol % to 99 mol % of 1-chloro-2,2-difluoroethane and from 99 mol % to 1 mol % of 1,1-dichloroethylene on the basis of the total composition thereof expressed in moles and the boiling point of the composition may be between 30° C. and 116° C.

### TECHNICAL FIELD

The present invention relates to compositions comprising 1-chloro-2,2-difluoroethane. In particular, the invention relates to compositions comprising 1-chloro-2,2-difluoroethane and 1,1-dichloroethylene. More particularly, the invention relates to azeotropic or quasi-azeotropic compositions comprising 1-chloro-2,2-difluoroethane and 1,1-dichloroethylene.

### TECHNOLOGICAL BACKGROUND OF THE INVENTION

Fluids based on halocarbons have found numerous applications in varied industrial fields, in particular as heat-transfer fluid, propellants, foaming agents, blowing agents, gaseous dielectrics, monomer or polymerization medium, support fluids, agents for abrasives, drying agents and fluids for energy production units. Document WO 2015/082812 describes a process for producing 1-chloro-2,2-difluoroethane.

The advantage of using azeotropic or quasi-azeotropic fluids lies in the absence of fractionation during the evaporation process and in the fact that they act (virtually) like a pure substance. However, it is difficult to identify new fluids satisfying these characteristics, since azeotropes are not predictable.

### SUMMARY OF THE INVENTION

The present invention provides an azeotropic or quasi-azeotropic composition comprising 1-chloro-2,2-difluoroethane and 1,1-dichloroethylene.

According to one preferred embodiment, said composition comprises from 1 mol % to 99 mol % of 1-chloro-2,2-difluoroethane and from 99 mol % to 1 mol % of 1,1-dichloroethylene on the basis of the total composition thereof expressed in moles.

According to one preferred embodiment, the boiling point of said composition is between 30° C. and 116° C.

According to one preferred embodiment, the pressure is between 1 and 11 bara.

According to one preferred embodiment, said composition comprises from 37 mol % to 67 mol % of 1-chloro-2,2-difluoroethane and from 33 mol % to 63 mol % of 1,1-dichloroethylene on the basis of the total composition thereof expressed in moles. Preferably, the composition is azeotropic.

According to one preferred embodiment, said composition comprises trans-1,2-dichloroethylene.

According to one preferred embodiment, said composition consists of 1-chloro-2,2-difluoroethane and 1,1-dichloroethylene.

According to one preferred embodiment, said composition consists of 1-chloro-2,2-difluoroethane, 1,1-dichloroethylene and trans-1,2-dichloroethylene.

According to one preferred embodiment, the molar ratio between the 1-chloro-2,2-difluoroethane and the trans-1,2-dichloroethylene is between 3 and 30.

According to a second aspect, the present invention provides a process for producing a composition comprising 1-chloro-2,2-difluoroethane and 1,1-dichloroethylene, comprising (i) at least one step during which the 1,1,2-trichloroethane reacts with hydrofluoric acid in the gas phase, optionally in the presence of an oxidizing agent, and in the presence or absence of a fluorination catalyst, to give a stream comprising 1-chloro-2,2-difluoroethane, hydrochloric acid, hydrofluoric acid and at least one compound A chosen from 1,2-dichloroethylenes (cis and trans), 1-chloro-2-fluoroethylenes (cis and trans), 1,2-dichloro-2-fluoroethane and unreacted 1,1,2-trichloroethane; (ii) at least one step of separating the compounds resulting from the reaction step, to give a first stream comprising hydrochloric acid and a second stream comprising hydrofluoric acid, 1-chloro-2,2-difluoroethane, at least one compound A and unreacted 1,1,2-trifluoroethane; (iii) at least one step of separating the second stream, to give an organic phase P1 comprising the 1-chloro-2,2-difluoroethane, at least one compound A and unreacted 1,1,2-trichloroethane and a nonorganic phase P2 comprising HF; (iv) at least one step of purifying the organic phase P1 obtained in (iii); characterized in that step (iv) comprises:
a) washing the organic phase P1 obtained in step (iii) so as to form an organic phase B1 comprising 1-chloro-2,2-difluoroethane, at least one compound A, unreacted 1,1,2-trichloroethane and 1,1-dichloroethylene; and a nonorganic phase B2 comprising hydrofluoric acid;
b) optionally, drying the organic phase B1 obtained in step a) so as to form an organic phase B3 comprising 1-chloro-2,2-difluoroethane, at least one compound A, unreacted 1,1,2-trichloroethane and 1,1-dichloroethylene;
c) purifying, preferably by distillation, the organic phase B1 or the organic phase B3 so as to form a stream B4 comprising 1-chloro-2,2-difluoroethane and 1,1-dichloroethylene; and an organic phase B5 comprising the unreacted 1,1,2-trichloroethane and at least one compound A.

According to one preferred embodiment, the washing step a) is carried out with water and the nonorganic phase B2 is an aqueous phase.

According to one preferred embodiment, step a) is carried out at a temperature of between 0° C. and 30° C. at a pressure of between 1 and 4 bara.

### DETAILED DESCRIPTION OF THE INVENTION

The present invention relates to a composition comprising 1-chloro-2,2-difluoroethane and 1,1-dichloroethylene. Preferably, said composition is azeotropic or quasi-azeotropic.

The expression “quasi-azeotropic” has a broad meaning and is intended to include compositions which are strictly azeotropic and those which behave like an azeotropic mixture.

The volatility of a compound A is represented by the ratio of the molar fraction in the gas phase (y_{A}) to the molar fraction in the liquid phase (x_{A}) under equilibrium conditions (at pressure and temperature equilibrium): α=y_{A}/x_{A}. The volatility of a compound B is represented by the ratio of the molar fraction in the gas phase (y_{B}) to the molar fraction in the liquid phase (x_{B}) under equilibrium conditions (at pressure and temperature equilibrium): α=y_{B}/x_{B}. The relative volatility makes it possible to measure the ease of separation of two compounds A and B. It is the ratio of the volatilities of the 2 compounds: α_{A,B}=y_{A}x_{B}/x_{A}y_{B}. The greater the volatility, the more the mixture can be easily separated.

When the relative volatility is equal to 1 or between 0.95 and 1.05, this means that the mixture is azeotropic. When the relative volatility is between 0.85 and 1.15, this means that the mixture is quasi-azeotropic.

Said composition may comprise from 1 mol % to 99 mol % of 1-chloro-2,2-difluoroethane on the basis of the total composition thereof expressed in moles. Preferably, said composition may comprise 1 mol % of 1-chloro-2,2-difluoroethane, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, 15 mol %, 16 mol %, 17 mol %, 18 mol %, 19 mol %, 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, 55 mol %, 56 mol %, 57 mol %, 58 mol %, 59 mol %, 60 mol %, 61 mol %, 62 mol %, 63 mol %, 64 mol %, 65 mol %, 66 mol %, 67 mol %, 68 mol %, 69 mol %, 70 mol %, 71mol %, 72 mol %, 73 mol %, 74 mol %, 75 mol %, 76 mol %, 77 mol %, 78 mol %, 79 mol %, 80 mol %, 81 mol %, 82 mol %, 83 mol %, 84 mol %, 85 mol %, 86 mol %, 87 mol %, 88 mol %, 89 mol %, 90 mol %, 91 mol %, 92 mol %, 93 mol %, 94 mol %, 95 mol %, 96 mol %, 97 mol %, 98 mol % or 99 mol % of 1-chloro-2,2-difluoroethane on the basis of the total composition thereof expressed in moles.

Said composition may comprise from 1 mol % to 99 mol % of 1,1-dichloroethylene on the basis of the total composition expressed in moles. Preferably, said composition may comprise 1 mol % of 1,1-dichloroethylene, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, 15 mol %, 16 mol %, 17 mol %, 18 mol %, 19 mol %, 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, 55 mol %, 56 mol %, 57 mol %, 58 mol %, 59 mol %, 60 mol %, 61 mol %, 62 mol %, 63 mol %, 64 mol %, 65 mol %, 66 mol %, 67 mol %, 68 mol %, 69 mol %, 70 mol %, 71 mol %, 72 mol %, 73 mol %, 74 mol %, 75 mol %, 76 mol %, 77 mol %, 78 mol %, 79 mol %, 80 mol %, 81 mol %, 82 mol %, 83 mol %, 84 mol %, 85 mol %, 86 mol %, 87 mol %, 88 mol %, 89 mol %, 90 mol %, 91 mol %, 92 mol %, 93 mol %, 94 mol %, 95 mol %, 96 mol %, 97 mol %, 98 mol % or 99 mol % of 1,1-dichloroethylene on the basis of the total composition thereof expressed in moles.

More preferentially, said composition may comprise 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, 15 mol %, 16 mol %, 17 mol %, 18 mol %, 19 mol %, 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, 55 mol %, 56 mol %, 57 mol %, 58 mol %, 59 mol %, 60 mol %, 61 mol %, 62 mol %, 63 mol %, 64 mol %, 65 mol %, 66 mol %, 67 mol %, 68 mol %, 69 mol %, 70 mol %, 71 mol %, 72 mol %, 73 mol %, 74 mol %, 75 mol %, 76 mol %, 77 mol %, 78 mol %, 79 mol %, 80 mol %, 81 mol %, 82 mol %, 83 mol %, 84 mol %, 85 mol %, 86 mol %, 87 mol %, 88 mol %, 89 mol %, 90 mol %, 91 mol %, 92 mol %, 93 mol %, 94 mol %, 95 mol %, 96 mol %, 97 mol %, 98 mol % or 99 mol % of 1-chloro-2,2-difluoroethane and 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, 15 mol %, 16 mol %, 17 mol %, 18 mol %, 19 mol %, 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, 55 mol %, 56 mol %, 57 mol %, 58 mol %, 59 mol %, 60 mol %, 61 mol %, 62 mol %, 63 mol %, 64 mol %, 65 mol %, 66 mol %, 67 mol %, 68 mol %, 69 mol %, 70 mol %, 71 mol %, 72 mol %, 73 mol %, 74 mol %, 75 mol %, 76 mol %, 77 mol %, 78 mol %, 79 mol %, 80 mol %, 81 mol %, 82 mol %, 83 mol %, 84 mol %, 85 mol %, 86 mol %, 87 mol %, 88 mol %, 89 mol %, 90 mol %, 91 mol %, 92 mol %, 93 mol %, 94 mol %, 95 mol %, 96 mol %, 97 mol %, 98 mol % or 99 mol % of 1,1-dichloroethylene on the basis of the total composition thereof expressed in moles.

In particular, said composition may comprise from 2 mol % to 98 mol % of 1-chloro-2,2-difluoroethane, from 3 mol % to 97 mol %, from 4 mol % to 96 mol %, from 5 mol % to 95 mol %, from 6 mol % to 94 mol %, from 7 mol % to 93 mol %, from 8 mol % to 92 mol %, from 9 mol % to 91 mol %, from 10 mol % to 90 mol %, from 11 mol % to 89 mol %, from 12 mol % to 88 mol %, from 13 mol % to 87 mol %, from 14 mol % to 86 mol %, from 15 mol % to 85 mol %, from 16 mol % to 84 mol %, from 17 mol % to 83 mol %, from 18 mol % to 82 mol %, from 19 mol % to 81 mol %, from 20 mol % to 80 mol %, from 21 mol % to 79 mol %, from 22 mol % to 78 mol %, from 23 mol % to 77 mol %, from 24 mol % to 76 mol %, from 25 mol % to 75 mol %, from 26 mol % to 74 mol %, from 27 mol % to 73 mol %, from 28 mol % to 72 mol %, from 29 mol % to 71 mol %, from 30 mol % to 70 mol %, from 31 mol % to 69 mol %, from 32 mol % to 68 mol %, from 33 mol % to 67 mol %, from 34 mol % to 67 mol %, from 35 mol % to 67 mol %, from 36 mol % to 67 mol % or from 37 mol % to 67 mol % of 1-chloro-2,2-difluoroethane on the basis of the total composition thereof expressed in moles.

In particular, said composition may comprise from 2 mol % to 98 mol % of 1,1-dichloroethylene, from 3 mol % to 97 mol %, from 4 mol % to 96 mol %, from 5 mol % to 95 mol %, from 6 mol % to 94 mol %, from 7 mol % to 93 mol %, from 8 mol % to 92 mol %, from 9 mol % to 91 mol %, from 10 mol % to 90 mol %, from 11 mol % to 89 mol %, from 12 mol % to 88 mol %, from 13 mol % to 87 mol %, from 14 mol % to 86 mol %, from 15 mol % to 85 mol %, from 16 mol % to 84 mol %, from 17 mol % to 83 mol %, from 18 mol % to 82 mol %, from 19 mol % to 81 mol %, from 20 mol % to 80 mol %, from 21 mol % to 79 mol %, from 22 mol % to 78 mol %, from 23 mol % to 77 mol %, from 24 mol % to 76 mol %, from 25 mol % to 75 mol %, from 26 mol % to 74 mol %, from 27 mol % to 73 mol %, from 28 mol % to 72 mol %, from 29 mol % to 71 mol %, from 30 mol % to 70 mol %, from 31 mol % to 69 mol %, from 32 mol % to 68 mol %, from 33 mol % to 67 mol %, from 33 mol % to 66 mol %, from 33 mol % to 65 mol %, from 33 mol % to 64 mol % or from 33 mol % to 63 mol % of 1,1-dichloroethylene on the basis of the total composition thereof expressed in moles.

Thus, according to one particular embodiment of the present invention, said composition may comprise from 2 mol % to 98 mol %, from 3 mol % to 97 mol %, from 4 mol % to 96 mol %, from 5 mol % to 95 mol %, from 6 mol % to 94 mol %, from 7 mol % to 93 mol %, from 8 mol % to 92 mol %, from 9 mol % to 91 mol %, from 10 mol % to 90 mol %, from 11 mol % to 89 mol %, from 12 mol % to 88 mol %, from 13 mol % to 87 mol %, from 14 mol % to 86 mol %, from 15 mol % to 85 mol %, from 16 mol % to 84 mol %, from 17 mol % to 83 mol %, from 18 mol % to 82 mol %, from 19 mol % to 81 mol %, from 20 mol % to 80 mol %, from 21 mol % to 79 mol %, from 22 mol % to 78 mol %, from 23 mol % to 77 mol %, from 24 mol % to 76 mol %, from 25 mol % to 75 mol %, from 26 mol % to 74 mol %, from 27 mol % to 73 mol %, from 28 mol % to 72 mol %, from 29 mol % to 71 mol %, from 30 mol % to 70 mol %, from 31 mol % to 69 mol %, from 32 mol % to 68 mol %, from 33 mol % to 67 mol %, from 34 mol % to 66 mol %, from 34 mol % to 67 mol %, from 35 mol % to 67 mol %, from 36 mol % to 67 mol % or from 37 mol % to 67 mol % of 1-chloro-2,2-difluoroethane and from 2 mol % to 98 mol %, from 3 mol % to 97 mol %, from 4 mol % to 96 mol %, from 5 mol % to 95 mol %, from 6 mol % to 94 mol %, from 7 mol % to 93 mol %, from 8 mol % to 92 mol %, from 9 mol % to 91 mol %, from 10 mol % to 90 mol %, from 11 mol % to 89 mol %, from 12 mol % to 88 mol %, from 13 mol % to 87 mol %, from 14 mol % to 86 mol %, from 15 mol % to 85 mol %, from 16 mol % to 84 mol %, from 17 mol % to 83 mol %, from 18 mol % to 82 mol %, from 19 mol % to 81 mol %, from 20 mol % to 80 mol %, from 21 mol % to 79 mol %, from 22 mol % to 78 mol %, from 23 mol % to 77 mol %, from 24 mol % to 76 mol %, from 25 mol % to 75 mol %, from 26 mol % to 74 mol %, from 27 mol % to 73 mol %, from 28 mol % to 72 mol %, from 29 mol % to 71 mol %, from 30 mol % to 70 mol %, from 31 mol % to 69 mol %, from 32 mol % to 68 mol %, from 33 mol % to 67 mol %, from 33 mol % to 66 mol %, from 33 mol % to 65 mol %, from 33 mol % to 64 mol % or from 33 mol % to 63 mol % of 1,1-dichloroethylene on the basis of the total composition thereof expressed in moles.

Preferably, the boiling point of said composition is between −50° C. and 250° C., more preferentially between −20° C. and 185° C., in particular between 5° C. and 145° C. More particularly, the boiling point of said composition is between 30° C. and 116° C.

Preferably, the pressure is between 0.005 bar and 20 bar, more preferentially from 0.3 bar to 15 bar abs. In particular, the pressure is between 1 and 11 bar abs.

Thus, the boiling point of said composition is between −50° C. and 250° C., more preferentially between −20° C. and 185° C., in particular between 5° C. and 145° C., more particularly between 30° C. and 116° C. at a pressure between 0.005 bar and 20 bar, more preferentially from 0.3 bar to 15 bar, more particularly between 1 and 11 bar abs.

According to one preferred embodiment, said composition comprises from 37 mol % to 67 mol % of 1-chloro-2,2-difluoroethane and from 33 mol % to 63 mol % of 1,1-dichloroethylene on the basis of the total composition thereof expressed in moles.

Preferably, said composition comprises from 37 mol % to 67 mol % of 1-chloro-2,2-difluoroethane and from 33 mol % to 63 mol % of 1,1-dichloroethylene on the basis of the total composition thereof expressed in moles, which the boiling point of said composition is between 30° C. and 116° C. at a pressure between 1 and 11 bara. Preferably, said composition in the proportions and under the conditions expressed herein is azeotropic.

According to one particular embodiment, said composition consists of 1-chloro-2,2-difluoroethane and 1,1-dichloroethylene, in the proportions detailed above. The 1-chloro-2,2-difluoroethane and the 1,1-dichloroethylene can be separated by extractive distillation so as to form a composition of 1-chloro-2,2-difluoroethane of high purity.

According to one preferred embodiment, said composition may also comprise trans-1,2-dichloroethylene. When the composition comprises trans-1,2-dichloroethylene, the molar ratio between the 1-chloro-2,2-difluoroethane and the trans-1,2-dichloroethylene may be between 3 and 30. Thus, a ternary composition comprising, preferably consisting of, 1-chloro-2,2-difluoroethane, 1,1-dichloroethylene and trans-1,2- dichloroethylene is provided. The proportions, the boiling point and the pressure are as detailed above. Thus, advantageously, said composition comprises, preferably consists of:
from 2 mol % to 98 mol % of 1-chloro-2,2-difluoroethane, from 3 mol % to 97 mol %, from 4 mol % to 96 mol %, from 5 mol % to 95 mol %, from 6 mol % to 94 mol %, from 7 mol % to 93 mol %, from 8 mol % to 92 mol %, from 9 mol % to 91 mol %, from 10 mol % to 90 mol %, from 11 mol % to 89 mol %, from 12 mol % to 88 mol %, from 13 mol % to 87 mol %, from 14 mol % to 86 mol %, from 15 mol % to 85 mol %, from 16 mol % to 84 mol %, from 17 mol % to 83 mol %, from 18 mol % to 82 mol %, from 19 mol % to 81 mol %, from 20 mol % to 80 mol %, from 21 mol % to 79 mol %, from 22 mol % to 78 mol %, from 23 mol % to 77 mol %, from 24 mol % to 76 mol %, from 25 mol % to 75 mol %, from 26 mol % to 74 mol %, from 27 mol % to 73 mol %, from 28 mol % to 72 mol %, from 29 mol % to 71 mol %, from 30 mol % to 70 mol %, from 31 mol % to 69 mol %, from 32 mol % to 68 mol %, from 33 mol % to 67 mol %, from 34 mol % to 67mol %, from 35 mol % to 67 mol %, from 36 mol % to 67 mol % or from 37 mol % to 67 mol % of 1-chloro-2,2-difluoroethane;
from 2 mol % to 98 mol % of 1,1-dichloroethylene, from 3 mol % to 97 mol %, from 4 mol % to 96 mol %, for 5 mol % to 95 mol %, from 6 mol % to 94 mol %, from 7 mol % to 93 mol %, from 8 mol % to 92 mol %, from 9 mol % to 91 mol %, from 10 mol % to 90 mol %, from 11 mol % to 89 mol %, from 12 mol % to 88 mol %, from 13 mol % to 87 mol %, from 14 mol % to 86 mol %, from 15 mol % to 85 mol %, from 16 mol % to 84 mol %, from 17 mol % to 83 mol %, from 18 mol % to 82 mol %, from 19 mol % to 81 mol %, from 20 mol % to 80 mol %, from 21 mol % to 79 mol %, from 22 mol % to 78 mol %, from 23 mol % to 77 mol %, from 24 mol % to 76 mol %, from 25 mol % to 75 mol %, from 26 mol % to 74 mol %, from 27 mol % to 73 mol %, from 28 mol % to 72 mol %, from 29 mol % to 71 mol %, from 30 mol % to 70 mol %, from 31 mol % to 69 mol %, from 32 mol % to 68 mol %, from 33 mol % to 67 mol %, from 33 mol % to 66 mol %, from 33 mol % to 65 mol %, from 33 mol % to 64 mol % or from 33 mol % to 63 mol % of 1,1-dichloroethylene; and
trans-1,2-dichloroethylene, the molar ratio between the 1-chloro-2,2-difluoroethane and the trans-1,2-dichloroethylene may be between 3 and 30;

on the basis of the total composition thereof expressed in moles.

The boiling point of said composition comprising 1-chloro-2,2-difluoroethane, 1,1-dichloroethylene and trans-1,2-dichloroethylene is between −50° C. and 250° C., more preferentially between −20° C. and 185° C., in particular between 5° C. and 145° C., more particularly between 30° C. and 116° C. at a pressure between 0.005 bar and 20 bar, more preferentially from 0.3 bar to 15 bar, more particularly between 1 and 11 bar abs.

According to a second aspect of the present invention, a process for producing a composition comprising 1-chloro-2,2-difluoroethane and 1,1-dichloroethylene is provided.

Said process comprises (i) at least one step during which the 1,1,2-trichloroethane reacts with hydrofluoric acid in the gas phase, optionally in the presence of an oxidizing agent, and in the presence or absence of a fluorination catalyst, to give a stream comprising 1-chloro-2,2-difluoroethane, hydrochloric acid, hydrofluoric acid and at least one compound A chosen from 1,2-dichloroethylenes (cis and trans), 1-chloro-2-fluoroethylenes (cis and trans), 1,2-dichloro-2-fluoroethane and unreacted 1,1,2-trichloroethane; (ii) at least one step of separating the compounds resulting from the reaction step, to give a first stream comprising hydrochloric acid and a second stream comprising hydrofluoric acid, 1-chloro-2,2-difluoroethane, at least one compound A and unreacted 1,1,2-trifluoroethane; (iii) at least one step of separating the second stream, to give an organic phase P1 comprising the 1-chloro-2,2-difluoroethane, at least one compound A and unreacted 1,1,2-trichloroethane and a nonorganic phase P2 comprising HF; (iv) at least one step of purifying the organic phase P1 obtained in (iii); characterized in that step (iv) comprises:
a) washing the organic phase P1 obtained in step (iii) so as to form an organic phase B1 comprising 1-chloro-2,2-difluoroethane, at least one compound A, unreacted 1,1,2-trichloroethane and 1,1-dichloroethylene; and a nonorganic phase B2 comprising hydrofluoric acid;
b) optionally, drying the organic phase B1 obtained in step a) so as to form an organic phase B3 comprising 1-chloro-2,2-difluoroethane, at least one compound A, unreacted 1,1,2-trichloroethane and 1,1-dichloroethylene;
c) purifying, preferably by distillation, the organic phase B1 or the organic phase B3 so as to form a stream B4 comprising 1-chloro-2,2-difluoroethane and 1,1-dichloroethylene; and an organic phase B5 comprising the unreacted 1,1,2-trichloroethane and at least one compound A.

Use is preferably made of a catalyst in step (i) and advantageously in the presence of an oxidizing agent. The temperature of the reaction step is preferably between 150 and 400° C., advantageously between 200 and 350° C. The pressure at which the fluorination reaction is carried out is preferably between 1 and 30 bar absolute, advantageously between 3 and 20 bar absolute and more particularly between 3 and 15 bar.

The amount of hydrofluoric acid used in the reaction is preferably between 5 and 40 mol and advantageously between 10 and 30 mol per mole of HCC-140.

The contact time, defined as being the volume of catalyst/total volume flow rate of gas at the temperature and pressure of the reaction, may be between 2 and 200 seconds, preferably between 2 and 100 seconds, advantageously between 2 and 50 seconds.

The oxidizing agent, pure or mixed with nitrogen, may be chosen from oxygen and chlorine. Chlorine is preferably chosen.

The amount of oxidizing agent used is preferably between 0.01 mol % and 20 mol % per mole of F140, advantageously between 0.01 mol % and 0.2 mol % per mole of HCC-140.

An amount of oxidizing agent of between 1 mol % and 10 mol % relative to the F140 has given very promising results.

The catalyst used may be a bulk or supported catalyst. The catalyst may be based on a metal, in particular on a transition metal or an oxide, halide or oxyhalide derivative of such a metal. By way of example, mention may in particular be made of FeCl₃, chromium oxyfluoride, NiCl₂, CrF₃ and mixtures thereof.

By way of supported catalysts, mention may be made of those supported on carbon or based on magnesium, such as magnesium derivatives, in particular halides such as MgF₂ or magnesium oxyhalides, such as oxyfluorides, or based on aluminum such as alumina, activated alumina or aluminum derivatives, in particular halides, such as AlF₃ or aluminum oxyhalides, such as oxyfluoride.

The catalyst may also comprise cocatalysts chosen from Co, Zn, Mn, Mg, V, Mo, Te, Nb, Sb, Ta, P, Ni, Zr, Ti, Sn, Cu, Pd, Cd, Bi and rare earth metals, or mixtures thereof. When the catalyst is chromium-based, Ni, Mg and Zn are advantageously chosen as cocatalyst.

The cocatalyst/catalyst atomic ratio is preferably between 0.01 and 5.

Chromium-based catalysts are particularly preferred.

The catalyst used in the present invention may be prepared by co-precipitation of the corresponding salts, optionally in the presence of a support.

The catalyst may also be prepared by co-milling of the corresponding oxides.

Prior to the fluorination reaction, the catalyst is subjected to a step of activation with HF at a temperature preferably of between 100 and 450° C., advantageously of between 200 and 400° C. for a period of between 1 and 50 hours.

In addition to the HF treatment, the activation may be carried out in the presence of the oxidizing agent.

The activation steps may be carried out at atmospheric pressure or under a pressure up to 20 bar abs.

According to one preferred embodiment of the invention, the support may be produced from alumina with a high porosity. In a first step, the alumina is converted into aluminum fluoride, or into a mixture of aluminum fluoride and alumina, by fluorination using air and hydrofluoric acid, the degree of conversion of the alumina into aluminum fluoride depending essentially on the temperature at which the fluorination of the alumina is carried out (in general between 200° C. and 450° C., preferably between 250° C. and 400° C.). The support is then impregnated by means of aqueous solutions of chromium salts, nickel salts and optionally rare earth metal salts, or by means of aqueous solutions of chromic acid, of nickel salt or zinc salt, and optionally of salts or oxides of rare earths and of methanol (serving as chromium-reducing agent). As chromium, nickel or zinc salts and rare earth metal salts, use may be made of chlorides, or other salts, such as, for example, oxalates, formates, acetates, nitrates and sulfates or dichromate of nickel, and of rare earth metals, provided that these salts are soluble in the amount of water capable of being absorbed by the support.

The catalyst may also be produced by direct impregnation of the alumina (which in general is activated) by means of solutions of the chromium, nickel or zinc, and optionally rare earth metal, compounds mentioned above. In this case, the conversion of at least one portion (for example 70% or more) of the alumina into aluminum fluoride or aluminum oxyfluoride is carried out during the step of activating the metal of the catalyst.

The activated aluminas that can be used to produce the catalyst are well-known, commercially available products. They are generally produced by calcination of alumina hydrates (aluminum hydroxides) at a temperature of between 300° C. and 800° C. The (activated or non-activated) aluminas may contain large amounts of sodium (up to 1000 ppm) without this being detrimental to the catalytic performance levels.

The catalyst is preferably conditioned or activated, that is to say converted into constituents that are active and stable (under the reaction conditions), by means of a prior “activation” operation. This treatment can be carried out either “in situ” (in the fluorination reactor) or alternatively in a suitable device designed to withstand the activation conditions.

After impregnation of the support, the catalyst is dried at a temperature between 100° C. and 350° C., preferably 220° C. to 280° C. in the presence of air or nitrogen.

The dried catalyst is then activated in one or two steps with hydrofluoric acid, optionally in the presence of an oxidizing agent. The duration of this fluorination activation step may be between 6 and 100 hours and the temperature between 200 and 400° C.

Preferably, the separating step (ii) comprises at least one distillation, advantageously carried out at a temperature of between −60° and 120° C. and more particularly between −60 and 89° C. and an absolute pressure of between 3 and 20 bar abs, and advantageously between 3 and 11 bar abs.

In addition to the 1-chloro-2,2-difluoroethane, the hydrofluoric acid and the 1,1,2-trichloroethane, the organic phase obtained in step (iii) also comprises at least one of the compounds A selected from the group consisting of cis-1,2-dichloroethylene, trans-1,2-dichloroethylene, cis-1-chloro-2-fluoroethylene, 1,2-dichloro-1-fluoroethane and trans-1-chloro-2-fluoroethylene.

After the separation of the second stream in step (iii), the nonorganic phase obtained in (iii) preferably contains the majority of the HF initially present in the second stream compared with the organic phase also obtained in step (iii). The organic phase obtained in (iii) may contain hydrofluoric acid. The amount of hydrofluoric acid in the organic phase is less than the amount of hydrofluoric acid in the nonorganic phase. The molar ratio of the hydrofluoric acid present in the organic phase to the hydrofluoric acid present in the nonorganic phase is less than 1:10, preferably less than 1:50, in particular 1:100.

Preferably, the separating step (iii) comprises at least one decanting step, advantageously carried out at a temperature of between −20 and 60° C. and more particularly between −20 and 10° C.

Preferably, the washing step a) is carried out with water and the nonorganic phase B2 is an aqueous phase. The washing step a) allows the formation of 1,1-dichloroethylene. The latter is recovered in the organic phase B1. Preferably, step a) is carried out at a temperature of between 0° C. and 30° C. at a pressure of between 1 and 4 bar abs.

The organic phase B1 may contain H₂O, preferably in a low proportion. Preferably, the H₂O content in the organic phase B1 is less than 5% by weight on the basis of the total weight of the organic phase B1, more preferentially less than 3% by weight, in particular less than 1% by weight. Thus, the organic phase B1 may comprise 1-chloro-2,2-difluoroethane, at least one compound A, unreacted 1,1,2-trichloroethane, 1,1-dichloroethylene and H₂O.

Step b) of drying the organic phase B1 may be carried out at a temperature between 0° C. and 30° C. at a pressure of between 1 and 4 bar abs. The drying step b) makes it possible to reduce the water content in the organic phase B1 so as to form an organic phase B3 comprising 1-chloro-2,2-difluoroethane, at least one compound A, unreacted 1,1,2-trichloroethane and 1,1-dichloroethylene. Preferably, the organic phase B3 comprises less than 1000 ppm of H₂O, more preferentially less than 100 ppm of H₂O, in particular less than 10 ppm of H₂O. The drying can preferably be carried out on a molecular sieve. Alternatively, the drying can be carried out in the presence of zeolite or of absorbents known to those skilled in the art.

The purifying step c) is preferably a distillation. The distillation of the organic phase B1 or B3 can be carried out at a temperature of from 10 to 100° C., preferably from 20 to 90° C., more preferentially from 30 to 80° C., and at an absolute pressure of from 0.3 to 8 bar abs, preferably from 0.5 to 6 bar abs, more preferentially from 1 to 4 bar. The purifying step c) allows, preferably, the formation of an azeotropic or quasi-azeotropic composition comprising 1-chloro-2,2-difluoroethane and 1,1-dichloroethylene as described above.

Preferably, said at least one compound A comprises the trans-1,2-dichloroethylene and at least one other compound A selected from the group consisting of cis-1,2-dichloroethylene, cis-1-chloro-2-fluoroethylene, 1,2-dichloro-1-fluoroethane and trans-1-chloro-2-fluoroethylene. In particular, when the purifying step c) is a distillation, the trans-1,2-dichloroethylene is preferably contained in the stream B4, the latter thus comprising 1-chloro-2,2-difluoroethane, 1,1-dichloroethylene and trans-1,2-dichloroethylene. In this case, the organic phase B5 preferably comprises unreacted 1,1,2-trichloroethane and said at least one other compound A selected from the group consisting of cis-1,2-dichloroethylene, cis-1-chloro-2-fluoroethylene, 1,2-dichloro-1-fluoroethane and trans-1-chloro-2-fluoroethylene.

According to one preferred embodiment, the process also comprises a step of recycling the organic phase B5 to step (i).

According to one preferred embodiment, the process also comprises a step of recycling, to step (i), the nonorganic phase P2 resulting from step (iii). According to one embodiment, before the recycling to step (i), the nonorganic phase P2 obtained in (iii) is purified so that the HF content is greater than or equal to 90% by weight. Preferably, this purification comprises at least one distillation, advantageously carried out at a temperature between −23 and 46° C. and an absolute pressure between 0.3 and 3 bar abs.

### EXAMPLES

### Experimental Procedure:

The HCC-140 and optionally the 1,2-dichloroethylene and the HF are fed separately into a monotubular inconel reactor heated by means of a fluidized alumina bath. The pressure is adjusted by means of a control valve located at the reactor outlet. The gases from the reaction are analyzed by gas chromatography. The catalyst is first dried under a nitrogen stream at 250° C., then the nitrogen is gradually replaced with HF so as to terminate the activation with pure HF (0.5 mol/h) at 350° C. for 8 h.

### Example 1

55 g are activated as described above. The HCC-140, the HF and the chlorine are then supplied with an HCC-140/HF/chlorine molar ratio of 1:9:0.08 (17 g/h of HF), at 230° C., 11 bar abs, with a contact time of 54 s. The F142 yield is 60% after 5 h. After 100 h, the yield is 62%. The mixture obtained is treated to separate the hydrofluoric acid from the other compounds. The organic phase obtained comprises 1-chloro-2,2-difluoroethane, trans-1,2-dichloroethylene and unreacted 1,1,2-trichloroethane. This phase is washed with water at a temperature between 0 and 30° C. at a pressure between 1 and 4 bar abs. The organic phase contains residual hydrofluoric acid not eliminated during the preceding step. The organic phase comprises 1-chloro-2,2-difluoroethane, trans-1,2-dichloroethylene, 1,1-dichloroethylene and unreacted 1,1,2-trichloroethane. The organic phase is then dried and distilled as detailed in the present application, so as to form a composition comprising 1-chloro-2,2-difluoroethane, trans-1,2-dichloroethylene and 1,1-dichloroethylene.

1. An azeotropic or quasi-azeotropic composition comprising 1-chloro-2,2-difluoroethane and 1,1-dichloroethylene.

2. The composition as claimed in claim 1, comprising from 1 mol % to 99 mol % of 1-chloro-2,2-difluoroethane and from 99 mol % to 1 mol % of 1,1-dichloroethylene on the basis of the total composition thereof expressed in moles.

3. The composition as claimed in claim 1, wherein the boiling point of said composition is between 30° C. and 116° C.

4. The composition as claimed in claim 1, wherein the pressure is between 1 and 11 bara.

5. The composition as claimed in claim 1, wherein it comprises from 37 mol % to 67 mol % of 1-chloro-2,2-difluoroethane and from 33 mol % to 63 mol % of 1,1-dichloroethylene on the basis of the total composition thereof expressed in moles.

6. The composition as claimed in claim 1, wherein it comprises trans-1,2-dichloroethylene.

7. The composition as claimed in claim 1, wherein it consists of 1-chloro-2,2-difluoroethane and 1,1-dichloroethylene.

8. The composition as claimed in claim 1, wherein it consists of 1-chloro-2,2-difluoroethane, 1,1-dichloroethylene and trans-1,2-dichloroethylene.

9. The composition as claimed in claim 8, wherein the molar ratio between the 1-chloro-2,2-difluoroethane and the trans-1,2-dichloroethylene is between 3 and 30.

10. A process for producing a composition comprising 1-chloro-2,2-difluoroethane and 1,1-dichloroethylene, comprising
(i) at least one step during which the 1,1,2-trichloroethane reacts with hydrofluoric acid in the gas phase, optionally in the presence of an oxidizing agent, and in the presence or absence of a fluorination catalyst, to give a stream comprising 1-chloro-2,2-difluoroethane, hydrochloric acid, hydrofluoric acid and at least one compound A chosen from 1,2-dichloroethylenes (cis and trans), 1-chloro-2-fluoroethylenes (cis and trans), 1,2-dichloro-2-fluoroethane and unreacted 1,1,2-trichloroethane;
(ii) at least one step of separating the compounds resulting from the reaction step, to give a first stream comprising hydrochloric acid and a second stream comprising hydrofluoric acid, 1-chloro-2,2-difluoroethane, at least one compound A and unreacted 1,1,2-trifluoroethane;
(iii) at least one step of separating the second stream, to give an organic phase P1 comprising the 1-chloro-2,2-difluoroethane, at least one compound A and unreacted 1,1,2-trichloroethane and a nonorganic phase P2 comprising HF;
(iv) at least one step of purifying the organic phase P1 obtained in (iii);
wherein step (iv) comprises:
a) washing the organic phase P1 obtained in step (iii) so as to form an organic phase B1 comprising 1-chloro-2,2-difluoroethane, at least one compound A, unreacted 1,1,2-trichloroethane and 1,1-dichloroethylene; and a nonorganic phase B2 comprising hydrofluoric acid;
b) optionally, drying the organic phase B1 obtained in step a) so as to form an organic phase B3 comprising 1-chloro-2,2-difluoroethane, at least one compound A, unreacted 1,1,2-trichloroethane and 1,1-dichloroethylene;
c) purifying, preferably by distillation, the organic phase B1 or the organic phase B3 so as to form a stream B4 comprising 1-chloro-2,2-difluoroethane and 1,1-dichloroethylene; and an organic phase B5 comprising the unreacted 1,1,2-trichloroethane and at least one compound A.

11. The process as claimed in claim 10, wherein the washing step a) is carried out with water and the nonorganic phase B2 is an aqueous phase.

12. The process as claimed in claim 10, wherein step a) is carried out at a temperature of between 0° C. and 30° C. at a pressure of between 1 and 4 bar abs.
